(19) United States
(12) Patent Application Publication
(10) Pub. No.: US 2025/0366754 A1
(43) Pub. Date: Dec. 4, 2025

(54) MOVEABLE SHEATHING FOR MEDICAL LEAD WIRES

(71) Applicant: Rhythmlink International, LLC, Columbia, SC (US)

(72) Inventors: Elliott Chapman, Columbia, SC (US); Sabrina Hasty, Lexington, SC (US); Jonathan Peterson, Columbia, SC (US); Brian Petree, West Columbia, SC (US)

(73) Assignee: Rhythmlink International, LLC, Columbia, SC (US)

(21) Appl. No.: 19/228,238

(22) Filed: Jun. 4, 2025

Related U.S. Application Data
(60) Provisional application No. 63/655,676, filed on Jun. 4, 2024.

(51) Int. Cl. A61B 5/273; H01B 7/00; H01B 7/18

(52) U.S. Cl. CPC A61B 5/273; H01B 7/0045; H01B 7/1805

(57) ABSTRACT

An adjustable sheathing system is provided for securing and protecting medical lead wires, comprising a lead wire bundle with multiple lead wires, connectors, and electrodes. The system includes first and second end portions, each containing a length of the lead wires and incorporating a securing mechanism. A sheath is connected to both the first and second end portions, encasing the lead wires to provide protection and organization. The securing mechanisms within the end portions enable adjustable retention of the lead wires, enhancing stability and reducing strain on the connections. This configuration facilitates secure management of medical lead wires while maintaining flexibility and protection throughout their length.

### CROSS-REFERENCE TO RELATED APPLICATIONS

This application claims the benefit and priority of U.S. Provisional Patent Application No. 63/655,676, filed on Jun. 4, 2024, titled Moveable Sheathing, the contents of which are incorporated herein.

### FIELD

The present technology generally relates to systems, devices, and methods relating to sheathing for lead wires, more particularly to moveable sheathing for protecting and encasing medical lead wire bundles.

### BACKGROUND

One challenge during neuromonitoring or neurophysiological monitoring, for example during electroencephalogram (EEG) or intraoperative neurophysiological monitoring (IONM) procedures, is managing lead wires that are used to connect a patient to one or more machines. In some instances, the lead wires used can range from about 0.5m in length to over 3.0m in length. In some instances, the number of lead wires can range from a single lead wire to over 30 lead wires. As will be appreciated, patient and/or technician movement during a procedure can cause one or more lead wires to become tangled which can cause issues and inconvenience to the technician and further can create a disconnection between a patient and a machine, for instance is an electrode pulled from the patient, or a lead wire is pulled from the machine. Furthermore, some lead wires are often left unconnected, or not in use, and this remain open and susceptible to interference and contact.

There are different conventional methods of lead wire management used in the EEG and IONM fields. One method is carried out by wrapping the lead wires in a woven harness sheath which is secured to a group of lead wires with heat shrink on both ends. In this instance, the sheath collects the lead wires together in a group and prevents them from tangling. However, one drawback to the harness sheath is that it limits the free length of individual lead wires in the bundle that is available to a technician during a given procedure. For example, during an EEG or IONM procedure, electrodes are placed at several locations on a subject's head and/or body with each electrode being connected to a lead wire. As will be appreciated in the industry, there is a unique distance from each electrode location to a machine into which the lead wires plug into. With the variability that some electrodes are farther away from a machine than other electrodes and require more free-length of lead wire to properly place and connect those electrodes. Further, some lead wires may not be in use, and thus there is a need to protect and sheath those to prevent accidental signal noise and stray or errant lead wires.

Thus, there is a long-sought need to provide devices and methods of and for managing lead wires which can also enable changes in the free length of individual lead wires, and protection and encapsulation of not in use lead wires. The disclosure herein provides embodiments in devices, systems, and methods that provide for technological advances in the management of lead wires to reduce patient and technician error and harm.

### SUMMARY

In some aspects, the techniques described herein relate to an adjustable sheathing system for securing and protecting medical lead wires, including: a lead wire bundle including a plurality of lead wires, connectors, and electrodes; a first end portion and a second end portion, included of an inner diameter and an outer diameter, wherein the first and second end portions contain a length of the lead wires; a securing mechanism within the first and second end portion, wherein the securing mechanism controls pressure; and a sheath connected to the first end portion and the second end portion, wherein the sheath encases the lead wires.

In some aspects, the techniques described herein relate to a system, wherein the first and second end portion further contains the connectors.

In some aspects, the techniques described herein relate to a system, wherein the sheath further encases the connectors.

In some aspects, the techniques described herein relate to a system, further including a third end portion, wherein the third end portion is connected to a first sheath and a second sheath.

In some aspects, the techniques described herein relate to a system, wherein the sheath is included of a non-conductive composite material.

In some aspects, the techniques described herein relate to a system, wherein the securing mechanism includes a brake component of a tab and a sloped extended body, wherein the tab is pulled to decrease the pressure on the lead wires and pushed in either the first or second end portions to increase the pressure on the lead wires.

In some aspects, the techniques described herein relate to a system, wherein the securing mechanism is a pressure fit mechanism that drives a protrusion on the inner diameter of the first end portion and the second end portion into the lead wires to increase friction.

In some aspects, the techniques described herein relate to a system, wherein the securing mechanism includes a lever, a lever, and a pawl to secure the first and second end portion to the lead wires by a one-handed operation.

In some aspects, the techniques described herein relate to a system, further including a dividing wall within the first and second end portion, wherein the dividing wall receives the lead wires in a first half and a second set of lead wires in a second other half.

In some aspects, the techniques described herein relate to a system, wherein the securing mechanism further includes a twist lock that when twisted causes the inner diameter of either the first or second end portion to reduce in size increasing the pressure.

In some aspects, the techniques described herein relate to an adjustable sheathing method for securing and protecting medical lead wires, including: provisioning a lead wire bundle including a plurality of lead wires, connectors, and electrodes; installing a first end portion and a second end portion, included of an inner diameter and an outer diameter, wherein the first and second end portions contain a length of the lead wires; configuring a securing mechanism within the first and second end portion, wherein configuring adjusts the securing mechanism to either increase or decrease pressure; and adjusting a sheath connected to the first end portion and the second end portion, wherein the sheath encases the lead wires and adjusting the sheath is conducted by moving either the first end portion or the second end portion by adjusting the securing mechanism.

In some aspects, the techniques described herein relate to a method, wherein the first and second end portion further contains the connectors.

In some aspects, the techniques described herein relate to a method, wherein the sheath further encases the connectors.

In some aspects, the techniques described herein relate to a method, further including a third end portion, wherein the third end portion is connected to a first sheath and a second sheath.

In some aspects, the techniques described herein relate to a method, wherein the sheath is included of a non-conductive composite material.

In some aspects, the techniques described herein relate to a method, wherein the securing mechanism includes a brake component of a tab and a sloped extended body, wherein the tab is pulled to decrease pressure on the lead wires, and pushed in either the first or second end portions to increase pressure on the lead wires.

In some aspects, the techniques described herein relate to a method, wherein the securing mechanism is a pressure fit mechanism that drives a protrusion on the inner diameter of the first end portion and the second end portion into the lead wires to increase friction.

In some aspects, the techniques described herein relate to a method, wherein the securing mechanism includes a lever, a lever, and a pawl to secure the first and second end portion to the lead wires by a one-handed operation.

In some aspects, the techniques described herein relate to a method, further including configuring a dividing wall within the first and second end portion, wherein the dividing wall receives the lead wires in a first half and a second set of lead wires in a second half.

In some aspects, the techniques described herein relate to a method, wherein the securing mechanism further includes a twist lock that when twisted causes the inner diameter of either the first or second end portion to reduce in size, increasing the pressure.

### BRIEF DESCRIPTION OF THE DRAWINGS

Aspects of the present disclosure can be better understood with reference to the following drawings to illustrate various principles of the disclosure. The components in the drawings are not necessarily to scale, with emphasis instead being placed upon clearly illustrating the principles of the disclosure. In the drawings:

FIG. 1 illustrates an example movable sheath installed on a lead wire in accordance with some aspects of the technology described herein;

FIGS. 2A-2B illustrate views of example sheath end portions, in accordance with some aspects of the present technology;

FIGS. 3A-3B illustrates an example system and method of using a movable sheath to encapsulate lengths and extra lead wires, in accordance with some aspects of the technology described herein;

FIG. 4 illustrates an example exploded view of inside a sheath of a movable sheath in accordance with some aspects of the technology described herein;

FIGS. 5A-5B illustrates an example of a secure end portion with a divider for dividing lead wire bundles;

FIGS. 6A-6C illustrate various example securing mechanisms for securing an end portion to a bundle of lead wires; and

FIG. 7 illustrates an example securing mechanism on an end portion that can be operated by one hand.

### DETAILED DESCRIPTION

The subject matter of the present disclosure is described with specificity herein to meet statutory requirements. However, the description itself is not intended to limit the scope of this patent. Rather, the inventors have contemplated that the claimed subject matter might also be embodied in other ways, to include different aspects or steps or combinations of steps similar to the ones described in this document, in conjunction with other present or future technologies. Moreover, although the terms “step” and/or “block” can be used herein to connote different elements of methods employed, the terms should not be interpreted as implying any particular order among or between various steps disclosed herein unless and except when the order of individual steps is explicitly described.

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein. Accordingly, embodiments described herein can be understood more readily by reference to the following detailed description, examples, and figures. Elements, apparatus, and methods described herein, however, are not limited to the specific embodiments presented in the detailed description, examples, and figures. It should be recognized that the exemplary embodiments herein are merely illustrative of the principles of the invention. Numerous modifications and adaptations will be readily apparent to those of skill in the art without departing from the scope of the technology.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

### Definitions

“Movable” or “Movability” means being able to be relocated at either end of the sheath, and slid or adjusted along the entire length of a single lead wire or a bundle of lead wires;

“Sheathing” means a covering, a jacket, or a wrap that is used to protect, insulate, bundle, organize, or otherwise encapsulate the lead wires;

“End Portion” or “Secured End Portion” are the various embodiments providing a secure connection to lead wires, that either lock, adjust to, or friction fit onto lead wires to reduce movement, and that also securely hold an end of the sheathing;

“Lead Wires” mean a metal wire connected from the electric pole of an electronic component; and

“Electrode” means a conductive pad or portion that detects electrical signals from the skin.

### Example Use Cases

According to aspects of the technology described herein, a moveable sheath, or as referred to from time to time, an adjustable sheath is provided that is configured to allow or enable extension or reduction in lead wire free length while grouping lead wires together and protecting lead wires and lead wire ends from accidental damage. As will be appreciated by those of skill in the art, devices and methods described herein can improve workflow for technicians in the medical field during medical procedures, for example EEG and IONM procedures, and help prevent disconnection between a patient and a machine. For example, disconnection of electrodes from a patient, disconnection of a lead wire from an electrode, and/or disconnection of a lead wire from a machine or device.

According to some embodiments, a moveable sheath can comprise a sheath portion and one or more securing end portions, which in some instances can be movable. In some instances, a moveable sheath may comprise a first securing end portion and a second securing end portion, wherein one may be at a distal length and the other at a proximal length on a bundle of lead wires. A sheath portion may be comprised of a woven or nonwoven length, of which can be made of any material not inconsistent with aspects of the present technology, including composite materials (polymers), rubbers, and natural fibers, or a combination thereof. The sheath portion may further comprise non-conductive properties, fire- and flame-resistant properties, thermal resistance, and abrasion resistance, while also being imparted with compression fibers. Further, a moveable sheath may be secured to one or more lead wires via at least one end portion. According to some aspects, moveability can be imparted to the device or to an end portion by placing a tube (e.g. PVC tube, or tube comprising another polymeric, rubber, or natural fiber material or combinations thereof) beneath a covering, wrap, and/or heat shrink (i.e. thermoplastic polymer such as Polyolefin, PVC, or Fluoropolymer) which can cover the tube and a portion of the sheath, such as an end of the sheath, in some instances to attach the tube to the sheath portion. In some other aspects, an adhesive may be used to attach a securing and/or end portion to the sheath.

In some aspects, movability is imparted to a sheath by the securing end portions, or end caps. The end portions being able to release pressure from a mechanical fitment on the exterior of the lead wires, and allowing for movement along a length of the lead wires. The end portions further clasping or securing the sheathing, and allow for the expansion and constriction on lead wire bundles.

In some aspects, movability is imparted by a combination of a polymeric tube and securing end portions, wherein the polymeric tube provides a lower degree of friction, and the securing end portions allow for mechanical fitment against lead wire bundles.

In other aspects, increased movability is imparted to a sheath by placing a tube beneath the wrap, that is used to secure the sheath to one or more (for example a group or bundle) lead wires. In doing so the overall friction is reduced allowing for easier movement. As will be appreciated the tube (in some instances a rigid, or semi-rigid, or flexible tube) can be any length not inconsistent with the technical objectives of the present technology. According to some aspects, the tube can have a diameter which is small enough (i.e. the tube can be configured to a specific diameter) to hold onto the lead wires, and simultaneously large enough to move along a bundle (e.g. two or more) of lead wires. Accordingly, in some instances, the tube can be friction fit to a bundle of wires. In some instances, the size of the tube, or diameter of the tube, is dependent on the number of lead wires to be bundled by the movable sheath. In some instances, the tube can be gauged to accommodate a bundle from two lead wires to over thirty lead wires.

In some aspects, the sheathing can have an elasticity that enables a tube to accommodate a range of lead wires. In some aspects, a securing end portion can have multiple or varying diameters across its length, such as a stepped or bell curve to allow for a range of lead wires. In other aspects, a securing end portion can have a first end having a first diameter and a second end having a second diameter. In some aspects an additional wrap or heat shrink portion can extend beyond an end portion.

According to some additional or other aspects, a securing mechanism (e.g. an automatic brake, mechanical brake, mechanical fitment) can be incorporated into one or more securing ends, that is, incorporated into sheath end portions. A securing mechanism can be implemented to hold the sheath portion in place and reduce unwanted movement, and in some aspects the securing mechanism can be released by a one handed operation to move the sheath along a portion of lead wires. In other aspects, one end portion may be fixed and secured to lead wires, while the other possesses a securing mechanism to allow movement along the lead wires. In other configurations, both ends have securing mechanisms, allowing for movement of both end portions, which in turn can account for increasing the sheath size to accommodate additional lead wires.

According to some embodiments, a moveable sheath may comprise a splitter which can allow or enable a technician to move two or more bundles of lead wires independently of one another. For example, a first bundle (e.g. bundle A) may contain 5 lead wires and a second bundle (e.g. bundle B) may contain 10 lead wires, and the splitter can be implemented to allow the first bundle of lead wires to move independently of the second bundle of lead wires. In some aspects, a moveable sheath may comprise a first securing end portion (e.g. positioned toward a machine end, distal end), a second securing end portion (e.g. positioned at the first bundle of lead wires), and a third securing end portion (e.g. positioned at the second bundle of lead wires, proximal end).

According to some embodiments, a securing end portion, or for instance a tube of a securing portion may incorporate one or more grooves or lanes (i.e. rather than a smooth tube shape) which allow for the grouping of lead wires into the grooves or lanes which can enable improved organizational and workflow capabilities.

### With Reference to the Drawings

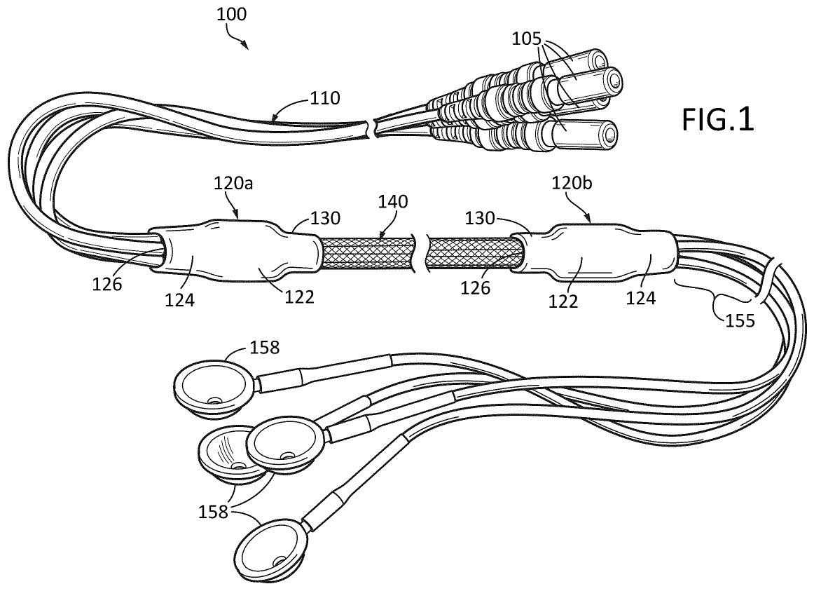

With reference to FIG. 1, an illustration of an example movable sheath installed on a lead wire in accordance with some aspects of the technology described herein. In the example lead wire connectors 105 are disclosed for connecting to machines for interpreting signals from the electrodes 158.

Continuing, in on aspect, the adjustable sheathing system 100 for securing and protecting medical lead wires includes components of a lead wire bundle 110 including a plurality of lead wires 155, connectors 105, and electrodes 158. Further, the adjustable sheathing system 100 comprises a first end portion 120a and a second end portion 120b. The end portions each having an inner diameter 126 and an outer diameter 124. The inner diameter being capable of a plurality of sizes and being restrained in place by a securing mechanism 122. The first and second end portions may comprise a length of the lead wires 110 and may also contain other medical equipment that may be configured alongside the lead wires 110. Further, the end portions may be wrapped in heat shrink, and the heat shrink may further serve as the binder to a sheath portion, the lead wires, or both. A securing mechanism 122 within the first and second end portion 120a, 120b, controls pressure or force asserted from the inner diameter 126 against the lead wires 110 to secure the respective end portion in place, in doing so also flexes by expanding or contracting the sheath 140. The sheath 140 is connected to the first end portion 120a and the second end portion 120b, wherein the sheath encases the lead wires to provide protection and to encapsulate lead wire bundles 155. The sheath 140, in some aspects, may be expandable with the addition of fibers that stretch and expand, such as Lycra, elastance, polyesters, nylons, and other natural fibers. The sheathing is typically comprised of non-conductive composite materials such as polymers. Further, there may be additional tubing accompanying the sheathing that provides greater resistance. The sheathing may also have a polymer or additive that reduces friction, for example polytetrafluorethylene, or silicone-based additives so that it may have less resistance against the lead wires, connectors, and other components that may be protected by the sheath. For example, a sheath may cover lead wires connected by connectors, thus the connectors are protected, encased, or otherwise contained within the sheath. In further examples, as third or fourth end portion may be equipped to traverse long lead wires with multiple connector groupings.

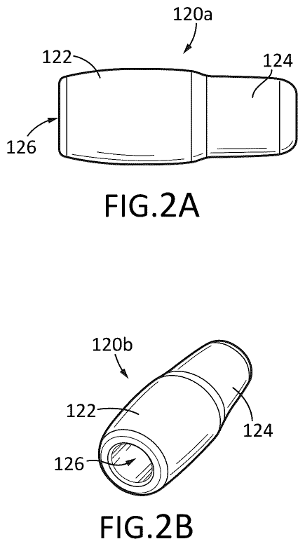

Referring to FIGS. 2A-2B, illustrating views of example end portions 120a 120b, in accordance with some aspects of the present technology. In the example, the lead wires enter on the first opening 126 and the sheath is secured on the second opening 124 by heat shrink, glue, adhesive, friction fit, compression, or other physical or chemical means of attachment to secure a composite or polymeric sleeve to the inner or outer diameter of the end portion. The outer diameter 122, in one aspect, is a hardened polymer that resist crushing and may have a securing mechanism built into the exterior that may cause pressure or a decrease in the size of the inner diameter based on the securing mechanism function. In some aspects, the securing mechanism further includes a twist lock that when twisted causes the inner diameter of either the first or second end portion 120a 120b to reduce in size increasing the pressure on lead wires. The securing mechanism allows for securing of the end portions against the lead wires and accounts for technician customization and protection of portions of a length of lead wires.

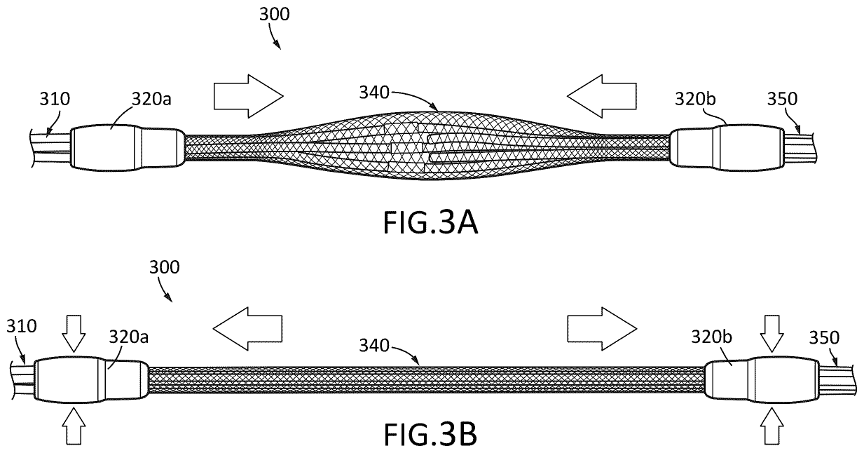

Referring now to FIGS. 3A-3B, illustrating an example system and method of using a movable sheath to encapsulate lengths and extra lead wires, in accordance with some aspects of the technology described herein. In the example, the sheath 340 is depicted in a compressed state in FIG. 3A, and a decompressed state FIG. 3B, and is adjusted by adjusting the end portions 320a 320b and securing mechanism against the lead wires 310, 350. In the example the sheath 340 is dynamic and capable of accommodating a plurality of lead wires or lead wire bundles, and may do so with a divided end portion that encapsulates the lead wire bundles into varying segments. Further, the sheath 340 provides insulation and may be used to “cap off” or otherwise contain lead wires not configured to connectors or electrodes, thus serving as an on-the-fly containment for unused lead wire ends. In further aspects, the sheath 340 provides insulation from noise and may be further secured to the end portions 320a, 320b by shrink wrap, compression fit, adhesives, or other means that secure the end portions 320a, 320b to the sheath 340.

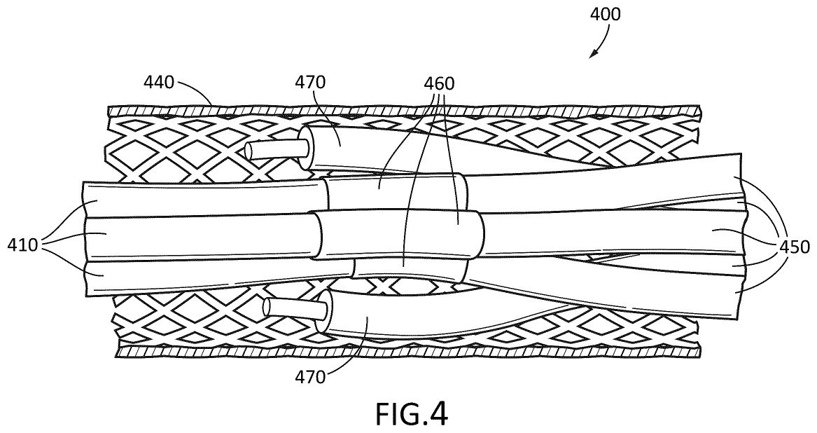

Referring now to FIG. 4, illustrating an example exploded view of inside a sheath of a movable sheath in accordance with some aspects of the technology described herein. In the example of FIG. 4, the lead wires 470 are contained within the sheath 440, and thus insulated from creating noise within the moveable sheathing system 400. The connected lead wires 410 may be connected via connectors 460, or shrink wrap, or may be continuous throughout and not be spliced together. This example depicts a method of splicing lead wires 410 and securing the splice with the moveable sheathing system 400, by encasing them in a non-conductive sheath 440. In the example polyethylene is used for the sheathing, in other examples polypropylene, or polystyrene, or polyvinyl chloride may be used, to name a few polymer or composite combinations.

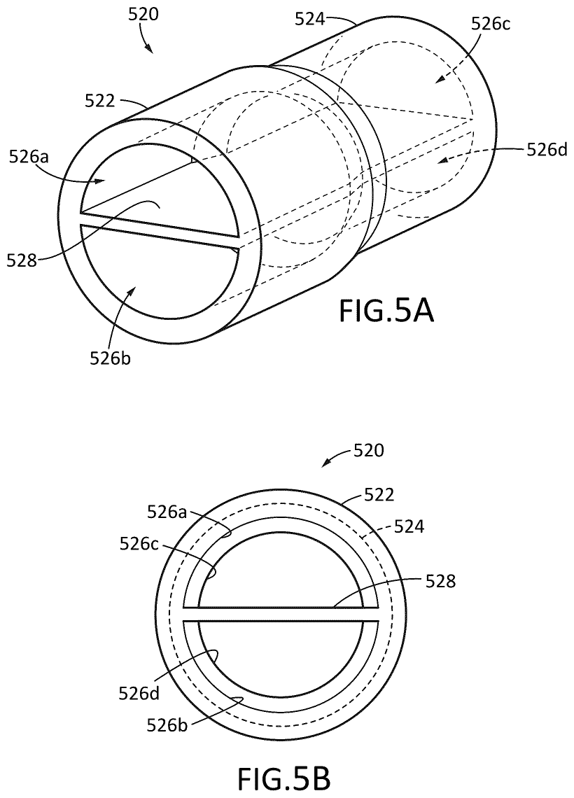

Continuing, FIGS. 5A-5B illustrate an example of a secure end portion 520 with a dividing wall 528 for dividing lead wire bundles. In some aspects, the techniques described herein relate to a system, further including a dividing wall 528 within the first and second end portion, wherein the dividing wall 528 receives the lead wires in a first half 526a and a second set of lead wires in a second other half 526b. In the example, the divider may assist in diving lead wire bundles belonging to different regions of acquiring electrical signals, or may be divided based on machine connectivity, or may be divided for ease of use of the technician or practitioner. In the example, the end portion 520 is comprised of a dividing wall 528, and a first half 526a, and a second half 526b, along with an outer diameter 522 of a lead wire input side and an outer diameter 524 of a sheath receptor side, along with attachment points 526c, 526d for sheathing to the end portion 520.

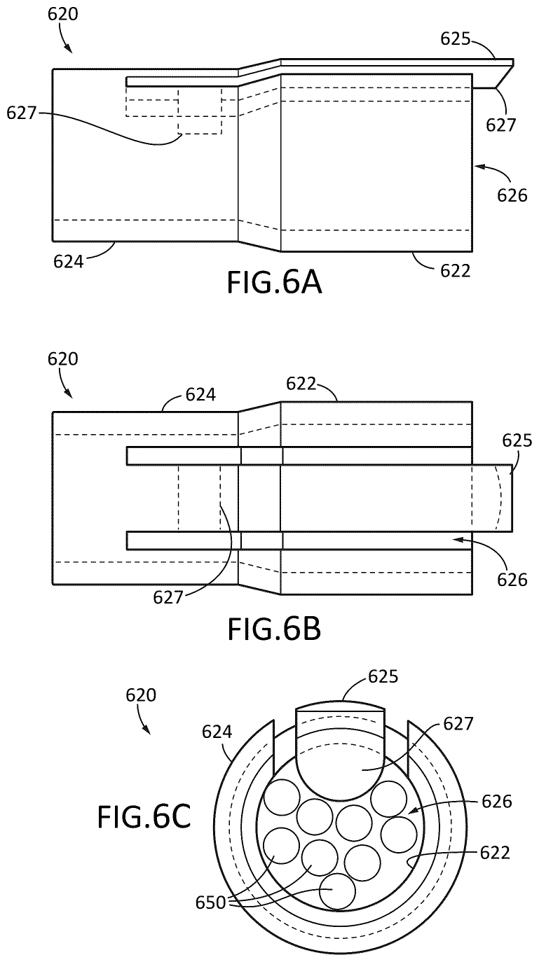

Referring now to FIGS. 6A-6C, illustrating various example securing mechanisms for securing an end portion 620 to a bundle of lead wires. In some aspects, the securing mechanism 625 includes a brake component 627 of a tab and a sloped extended body, that in some aspects may comprise a tab, wherein the tab is pulled to decrease the pressure on the lead wires, and pushed in either the first or second end portions to increase the pressure on the lead wires. In this example the outer diameter 624 remains unchanged, and the inner diameter is adjusted based on the brake component 627 and the tab associated therewith. Various aspects of this wedge style friction fit are disclosed, and the example in FIGS. 6A and 6B are not inclusive, the principles at work of the brake securing mechanism 625 are that of a friction wedge that can be adjusted to press firmly against lead wire bundles or be pulled to release the fit and allow for movement of the end portion 620. The end portion 620 having a fixed outer diameter 624, and an adjustable inner diameter based on how far the braking mechanism is subjected to the end portion 620.

Continuing, in FIG. 6C, wherein the securing mechanism is a pressure fit securing mechanism 620 that drives a protrusion 627 on the inner diameter 622 of the first end portion 624 and the second end portion into the lead wires 650 to increase friction and reduce the spacing 626 between the lead wires. In this aspect, the outer diameter and the inner diameter remain unchanged except for the protrusion 627, which can be operated by a one-hand mechanism of depressing the button 625 on the exterior of the end portion 624. The pressure fit securing mechanism 620 can be released by a subsequent hard press on the button 625, which releases a ratchet that allows for the protrusion 627 to move outward, thus providing an increase in the inner diameter 622 and allowing for greater spacing and less resistance from the lead wires 650.

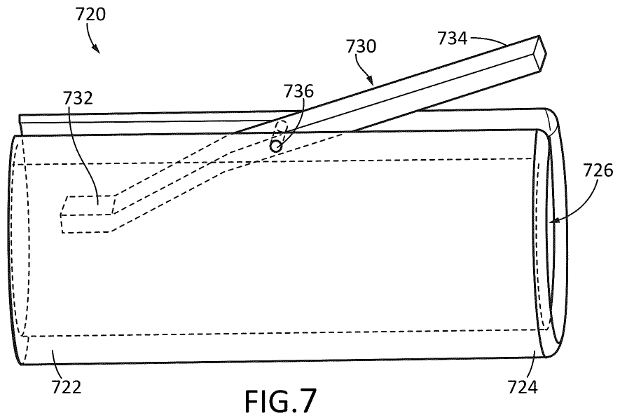

Referring now to FIG. 7, illustrating an example securing mechanism 720 on an end portion 722 that can be operated by one hand. In some aspects, the securing mechanism 720 includes a lever 734, a fulcrum 736, and a pawl 730 to secure the first and second end portion to the lead wires by a one-handed operation. In this example the prong 732 acts as against the lead wires with the fulcrum 736 aiding in increasing pressure when the lever 734 is listed. The pawl 730 aids in holding the pressure or resistance against the lead wires, thus securing the end portion 722 against the lead wires and reducing its ability to move. In this aspect, the inner diameter 726 remains unchanged, as does the outer diameter 724, allowing for the only change to arrive from the prong 732 being depressed into the inner diameter 726.

The above-described embodiments of the present disclosure are merely possible examples of implementations set forth for a clear understanding of the principles of the disclosure. Many variations and modifications may be made to the above-described embodiments without departing substantially from the scope and principles of the disclosure. All such modifications and variations are intended to be included herein within the scope of this disclosure and protected by the following claims.

Many different arrangements of the various components and/or steps depicted and described, as well as those not shown, are possible without departing from the scope of the claims below. Embodiments of the present technology have been described with the intent to be illustrative rather than restrictive. Alternative embodiments will become apparent from reference to this disclosure. Alternative means of implementing the can be completed without departing from the scope of the claims below. Certain features and sub combinations are of utility and can be employed without reference to other features and sub combinations and are contemplated within the scope of the claims.

Therefore, We claim:

1. An adjustable sheathing system for securing and protecting medical lead wires, comprising:
a lead wire bundle comprising a plurality of lead wires, connectors, and electrodes;
a first end portion and a second end portion, comprised of an inner diameter and an outer diameter, wherein the first and second end portions contain a length of the lead wires;
a securing mechanism within the first and second end portion, wherein the securing mechanism controls pressure; and
a sheath connected to the first end portion and the second end portion, wherein the sheath encases the lead wires.

2. The system of claim 1, wherein the first and second end portion further contains the connectors.

3. The system of claim 2, wherein the sheath further encases the connectors.

4. The system of claim 1, further comprising a third end portion, wherein the third end portion is connected to a first sheath and a second sheath.

5. The system of claim 1, wherein the sheath is comprised of a non-conductive composite material.

6. The system of claim 1, wherein the securing mechanism comprises a brake component of a tab and a sloped extended body, wherein the tab is pulled to decrease the pressure on the lead wires, and pushed in either the first or second end portions to increase the pressure on the lead wires.

7. The system of claim 1, wherein the securing mechanism is a pressure fit mechanism that drives a protrusion on the inner diameter of the first end portion and the second end portion into the lead wires to increase friction.

8. The system of claim 1, wherein the securing mechanism comprises a lever, a lever, and a pawl to secure the first and second end portion to the lead wires by a one-handed operation.

9. The system of claim 1, further comprising a dividing wall within the first and second end portion, wherein the dividing wall receives the lead wires in a first half and a second set of lead wires in a second other half.

10. The system of claim 1, wherein the securing mechanism further comprises a twist lock that when twisted causes the inner diameter of either the first or second end portion to reduce in size increasing the pressure.

11. An adjustable sheathing method for securing and protecting medical lead wires, comprising:
provisioning a lead wire bundle comprising a plurality of lead wires, connectors, and electrodes;
installing a first end portion and a second end portion, comprised of an inner diameter and an outer diameter, wherein the first and second end portions contain a length of the lead wires;
configuring a securing mechanism within the first and second end portion, wherein configuring adjusts the securing mechanism to either increase or decrease pressure; and
adjusting a sheath connected to the first end portion and the second end portion, wherein the sheath encases the lead wires and adjusting the sheath is conducted by moving either the first end portion or the second end portion by adjusting the securing mechanism.

12. The method of claim 11, wherein the first and second end portion further contains the connectors.

13. The method of claim 12, wherein the sheath further encases the connectors.

14. The method of claim 11, further comprising a third end portion, wherein the third end portion is connected to a first sheath and a second sheath.

15. The method of claim 11, wherein the sheath is comprised of a non-conductive composite material.

16. The method of claim 11, wherein the securing mechanism comprises a brake component of a tab and a sloped extended body, wherein the tab is pulled to decrease pressure on the lead wires, and pushed in either the first or second end portions to increase pressure on the lead wires.

17. The method of claim 11, wherein the securing mechanism is a pressure fit mechanism that drives a protrusion on the inner diameter of the first end portion and the second end portion into the lead wires to increase friction.

18. The method of claim 11, wherein the securing mechanism comprises a lever, a lever, and a pawl to secure the first and second end portion to the lead wires by a one-handed operation.

19. The method of claim 11, further comprising configuring a dividing wall within the first and second end portion, wherein the dividing wall receives the lead wires in a first half and a second set of lead wires in a second half.

20. The method of claim 11, wherein the securing mechanism further comprises a twist lock that when twisted causes the inner diameter of either the first or second end portion to reduce in size, increasing the pressure.
